(19) United States
(12) Patent Application Publication
(10) Pub. No.: US 2022/0211400 A1
(43) Pub. Date: Jul. 7, 2022

(54) DEVICE AND METHOD FOR TREATING VASCULAR OCCLUSION

(71) Applicant: Inari Medical, Inc., Irvine, CA (US)

(72) Inventors: Brian J. Cox, Laguna Niguel, CA (US); Paul Lubock, Monarch Beach, CA (US); Robert F. Rosenbluth, Laguna Niguel, CA (US)

(21) Appl. No.: 17/704,258

(22) Filed: Mar. 25, 2022

Related U.S. Application Data
(63) Continuation of application No. 15/978,560, filed on May 14, 2018.
Application No. 15/978,560 is a continuation of application No. 15/466,740, filed on Mar. 22, 2017, now Pat. No. 10,045,790.
Application No. 15/466,740 is a continuation of application No. 14/430,519, filed on Mar. 23, 2015.
(60) Provisional application No. 61/705,129, filed on Sep. 24, 2012.
(60) Provisional application No. 61/728,755, filed on Nov. 20, 2012.
(60) Provisional application No. 61/750,277, filed on Jan. 8, 2013.
(60) Provisional application No. 61/845,796, filed on Jul. 12, 2013.
(60) Provisional application No. 61/864,356, filed on Aug. 9, 2013.

(51) Int. Cl. A61B 17/221

(52) U.S. Cl. CPC A61B 17/221; A61B 2017/22034; F04C 2270/0421

(57) ABSTRACT

A system and method for managing an occlusion, such as a blood clot, within a lumen or passageway of a patient. More particularly, a system and method for rapidly restoring blood flow through an occlusion including a self-expanding, tubular member through which blood may flow when in an expanded state. The tubular member has a structure configured to engage the occlusive material, thereby allowing for extraction of at least a portion of the occlusive material. The system may further employ a material extraction member that is deployed distally of the tubular member.

### CROSS-REFERENCE TO RELATED APPLICATIONS

This application is a continuation of U.S. patent application Ser. No. 15/978,560, filed May 14, 2018, entitled Device and Method for Treating Vascular Occlusion, which is a continuation of U.S. patent application Ser. No. 15/466,740, filed Mar. 22, 2017, entitled Device and Method for Treating Vascular Occlusion, now issued as U.S. Pat. No. 10,045,790, which is a continuation of U.S. patent application Ser. No. 14/430,519, filed on Mar. 23, 2015, entitled Device and Method for Treating Vascular Occlusion, which is a 35 U.S.C. 371 of International Patent Application No. PCT/US2013/061470, filed on Sep. 24, 2013, entitled Device and Method for Treating Vascular Occlusion, which claims priority to U.S. Provisional Application No. 61/864,356, filed Aug. 9, 2013, entitled Devices and Methods for Treatment of Vascular Occlusion, U.S. Provisional Application No. 61/845,796, filed Jul. 12, 2013, entitled Devices and Methods for Treatment of Vascular Occlusion, U.S. Provisional Application No. 61/750,277, filed Jan. 8, 2013, entitled Devices and Methods for Treatment of Vascular Occlusion, U.S. Provisional Application No. 61/728,775, filed Nov. 20, 2012, entitled Devices and Methods for Treatment of Vascular Occlusion; and U.S. Provisional Application No. 61/705,129, filed Sep. 24, 2012, entitled Devices and Methods for Treatment of Vascular Occlusion, each of which are incorporated herein by reference in their entireties.

### TECHNICAL FIELD

This invention relates to a system and method for endovascular treatment of blood clots obstructing passageways in the circulatory system.

### BACKGROUND

Thromboembolism is the formation in a blood vessel of a clot (thrombus) that breaks loose (embolizes) and is carried by the blood stream to another location in the circulatory system resulting in a clot or obstruction at that new location. For example, a clot may embolize and plug a vessel in the lungs (pulmonary embolism), the brain (stroke), the gastrointestinal tract, the kidneys, or the legs. Thromboembolism is a significant cause of morbidity (disease) and mortality (death), especially in adults. A thromboembolism can be sudden and massive or it may be small and multiple. A thromboembolism can be any size and a thromboembolic event can happen at any time.

When a thrombus forms in the venous circulation of the body it often embolizes to the lungs. Such a thrombus typically embolizes from the veins of the legs, pelvis, or inferior vena cava and travels to the right heart cavities and then into the pulmonary arteries thus resulting in a pulmonary embolism.

A pulmonary embolism results in right heart failure and decreased blood flow through the lungs with subsequent decreased oxygenation of the lungs, heart and the rest of the body. More specifically, when such a thrombus enters the pulmonary arteries, obstruction and spasm of the different arteries of the lung occurs which further decreases blood flow and gaseous exchange through the lung tissue resulting in pulmonary edema. All of these factors decrease the oxygen in the blood in the left heart. As a result, the oxygenated blood supplied by the coronary arteries to the musculature of both the left and right heart is insufficient for proper contractions of the muscle which further decreases the entire oxygenated blood flow to the rest of the body. This often leads to heart dysfunction and specifically right ventricle dysfunction.

This condition is relatively common and has many causes. Some of the more common causes are prolonged inactivity such as bed rest, extended sitting (e.g., lengthy aircraft travel), dehydration, extensive surgery or protracted disease. Almost all of these causes are characterized by the blood of the inferior peripheral major circulatory system coagulating to varying degrees and resulting in permanent drainage problems.

There exist a number of approaches to treating thromboembolism and particularly pulmonary embolism. Some of those approaches include the use of anticoagulants, thrombolytics and endovascular attempts at removal of the emboli from the pulmonary artery. The endovascular attempts often rely on catheterization of the affected vessels and application of chemical or mechanical agents or both to disintegrate the clot. Invasive surgical intervention in which the emboli is removed by accessing the chest cavity, opening the embolized pulmonary artery and/or its branches and removing the clot is also possible.

The prior approaches to treatment, however, are lacking. For example, the use of agents such as anticoagulants and/or thrombolytics to reduce or remove a pulmonary embolism typically takes a prolonged period of time, e.g., hours and even days, before the treatment is effective. Moreover, such agents can cause hemorrhage in a patient.

And the known mechanical devices for removing an embolism are typically highly complex and prone to cause undue trauma to the vessel. Moreover, such known devices are difficult and expensive to manufacture.

Lastly, the known treatment methods do not emphasize sufficiently the goal of urgently restoring blood flow through the thrombus once the thrombus has been identified. In other words, the known methods focus primarily and firstly on overall clot reduction and removal instead of first focusing on relief of the acute blockage condition followed then by the goal of clot reduction and removal. Hence, known methods are not providing optimal patient care, particularly as such care relates to treatment of a pulmonary embolism.

### SUMMARY

The above described shortcomings of the existing systems and approaches for treating an occlusion in a lumen of a patient, such as a thromboembolism and particularly a pulmonary embolism, are improved upon by the systems and methods of the present invention. These improvements are achieved in certain embodiments of the present invention, in part, by providing an occlusion management system comprising a catheter, a pusher, and a tubular member reversibly restrained in a compressed state within a lumen of the catheter and radially expanded from the compressed state upon retraction of the catheter relative to the pusher.

These improvements are further achieved in certain embodiments of the present invention, in part, by providing occlusion management system comprising a catheter, a pusher, a tubular member attached to a distal end of the pusher, and an extraction member extending distally of a distal end of the cylindrical member having a diameter larger than a diameter of the cylindrical member.

These improvements are further achieved in certain embodiments of the present invention, in part, by a method for management of an occlusion in a lumen comprising the steps of: creating a passage for fluid flow through occlusive material in a lumen of a patient, engaging a portion of the occlusive material with at least a portion of a tubular member; and extracting a portion of the occlusive material from the lumen of the patient.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a partial cutaway view of a portion of an occlusion management system within a lumen of a patient according to one embodiment of the present invention.

FIG. 2 is a partial cutaway view of a portion of an occlusion management system within a lumen of a patient according to one embodiment of the present invention.

FIG. 3 is a partial cutaway view of a portion of an occlusion management system within a lumen of a patient according to one embodiment of the present invention.

FIG. 4 is a partial cutaway view of a portion of an occlusion management system within a lumen of a patient according to one embodiment of the present invention.

FIG. 5 is a partial cutaway view of a portion of an occlusion management system within a lumen of a patient according to one embodiment of the present invention.

FIG. 6 is a partial cutaway view of a portion of an occlusion management system within a lumen of a patient according to one embodiment of the present invention.

FIG. 7 is a side elevation view of a portion of an occlusion management system according to one embodiment of the present invention.

FIG. 8 is a partial cutaway view of a portion of an occlusion management system within a lumen of a patient according to one embodiment of the present invention.

FIG. 9 is a partial cutaway view of a portion of an occlusion management system within a lumen of a patient according to one embodiment of the present invention.

FIG. 10 is a partial cutaway view of a portion of an occlusion management system within a lumen of a patient according to one embodiment of the present invention.

FIG. 11 is a partial cutaway view of a portion of an occlusion management system within a lumen of a patient according to one embodiment of the present invention.

FIG. 12 is a partial cutaway elevation view of a portion of an occlusion management system according to one embodiment of the present invention.

FIG. 13 is a partial cutaway view of a portion of an occlusion management system within a lumen of a patient according to one embodiment of the present invention.

FIG. 14 is a partial cutaway view of a portion of an occlusion management system within a lumen of a patient according to one embodiment of the present invention.

FIG. 15 is a partial cutaway view of a portion of an occlusion management system within a lumen of a patient according to one embodiment of the present invention.

FIG. 16 is a partial cutaway view of a portion of an occlusion management system within a lumen of a patient according to one embodiment of the present invention.

FIG. 17 is a partial cutaway view of a portion of an occlusion management system according to one embodiment of the present invention.

FIG. 18 is a partial cutaway view of a portion of an occlusion management system according to one embodiment of the present invention.

FIG. 19 is a partial cutaway view of a portion of an occlusion management system within a lumen of a patient according to one embodiment of the present invention.

FIG. 20 is a partial cutaway view of a portion of an occlusion management system within a lumen of a patient according to one embodiment of the present invention.

FIG. 21 is a partial cutaway view of a portion of an occlusion management system within a lumen of a patient according to one embodiment of the present invention.

FIG. 22 is a partial cutaway view of a portion of an occlusion management system within a lumen of a patient according to one embodiment of the present invention.

FIG. 23 is a partial cutaway view of a portion of an occlusion management system within a lumen of a patient according to one embodiment of the present invention.

FIG. 24 is a partial cutaway view of a portion of an occlusion management system within a lumen of a patient according to one embodiment of the present invention.

FIG. 25 is a partial cutaway view of a portion of an occlusion management system within a lumen of a patient according to one embodiment of the present invention.

FIG. 26 is a partial cutaway view of a portion of an occlusion management system within a lumen of a patient according to one embodiment of the present invention.

FIG. 27 is a partial cutaway view of a portion of an occlusion management system within a lumen of a patient according to one embodiment of the present invention.

FIG. 28A is an elevation view of a portion of an occlusion management system according to one embodiment of the present invention.

FIG. 28B is a partial cutaway view of a portion of an occlusion management system within lumen of a patient according to one embodiment of the present invention.

FIG. 29 is a partial cutaway view of a portion of an occlusion management system within a lumen of a patient according to one embodiment of the present invention.

FIG. 30 is a partial cutaway view of a portion of an occlusion management system within a lumen of a patient according to one embodiment of the present invention.

FIG. 31 is a partial cutaway view of a portion of an occlusion management system within a lumen of a patient according to one embodiment of the present invention.

FIG. 32 is a partial cutaway view of a portion of an occlusion management system within a lumen of a patient according to one embodiment of the present invention.

FIG. 33 is a partial cutaway view of a portion of an occlusion management system within a lumen of a patient according to one embodiment of the present invention.

FIG. 34 is a partial cutaway view of a portion of an occlusion management system within a lumen of a patient according to one embodiment of the present invention.

FIG. 35 is a partial cutaway view of a portion of an occlusion management system within a lumen of a patient according to one embodiment of the present invention.

FIG. 36 is a partial cutaway view of a portion of an occlusion management system within a lumen of a patient according to one embodiment of the present invention.

FIG. 37 is a partial cutaway view of a portion of an occlusion management system within a lumen of a patient according to one embodiment of the present invention.

FIG. 38 is an elevation view of a portion of an occlusion management system according to one embodiment of the present invention.

FIG. 39 is a perspective view of a portion of an occlusion management system according to one embodiment of the present invention.

FIG. 40A-40C are elevation views of portions of an occlusion management system according to one embodiment of the present invention.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Methods and systems according to the present invention are broadly directed to treating a blood vessel or other body lumen. More particularly, the present invention is directed to systems and methods for disrupting, dissolving, and/or otherwise removing occlusive materials, such as thrombus, from a treatment site, such as a blood vessel.

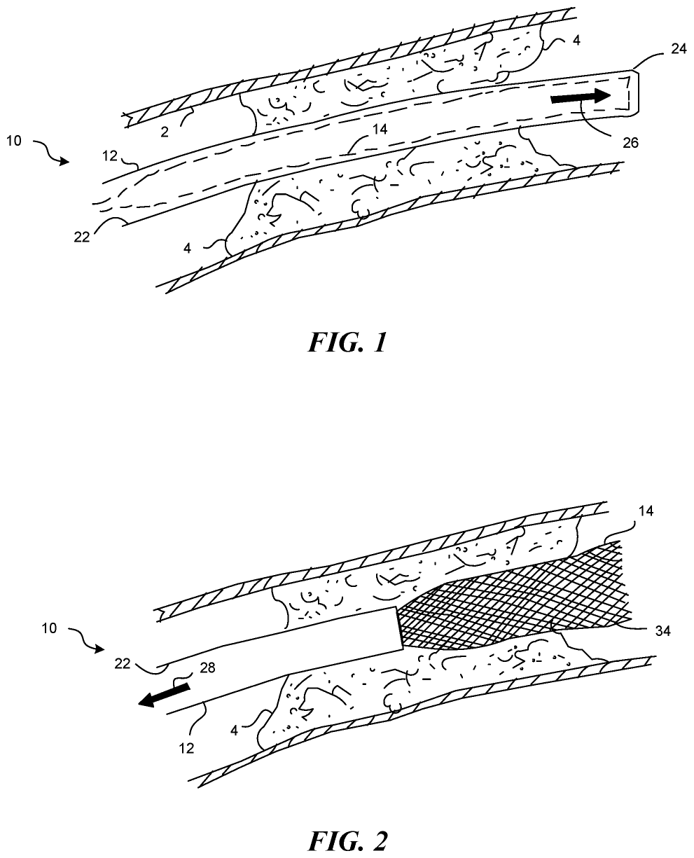

With reference to FIGS. 1-6, in one embodiment of the present invention, an occlusion management system 10 employs a catheter 12 and a flow restoration member 14. The flow restoration member 14 is radially expandable from a compressed delivery state, to a radially expanded, minimum energy state having at least, in part, a hollow cylindrical or tubular shape. A distal end 18 of a pusher 16 is attached to a proximal portion 20 of the flow restoration member 14.

The flow restoration member 14 may be formed of a porous mesh or scaffold. The mesh or scaffold may be formed at least in part by a braid of filaments or fabricated by methods known in the art of stent manufacturing including but not limited to conventional machining, laser cutting, electrical discharge machining (EDM) and photochemical etching.

In operation, the pusher 16 and the attached compressed flow restoration member 14 are inserted into a lumen 22 of the catheter 12. The catheter 12 is advanced through a lumen 2 of a patient, e.g. a blood vessel 2, to a site within the lumen 2 at which occlusive material 4, such as a thrombus or an embolus, is located. The catheter 12 is advanced in the direction of arrow 26 through the occlusive material 4 until a distal end 24 of the catheter 12 passes entirely through the occlusive material 4, as shown in FIG. 1.

With reference to FIG. 2, the catheter 12 is then retracted relative to the pusher 16 and flow restoration member 14 in the direction of arrow 28. As the flow restoration member 14 is exposed from the retracting distal end of the catheter 12, the flow restoration member 14 radially expands within the occlusive material 4 to an intermediate diameter larger than a diameter of the member 14 in the compressed delivery state and smaller than a diameter of the member 14 in the expanded, minimum energy state. The structure and outer surface of the flow restoration member 14 is configured such that the mesh or scaffold of the flow restoration member 14 engages the occlusive material 4 when it is exposed from the constraint of the catheter 12. As shown in FIG. 2, the catheter 12 is retracted in the direction of arrow 28 to an extent that allows for the radial expansion of an entire length of the flow restoration member 14.

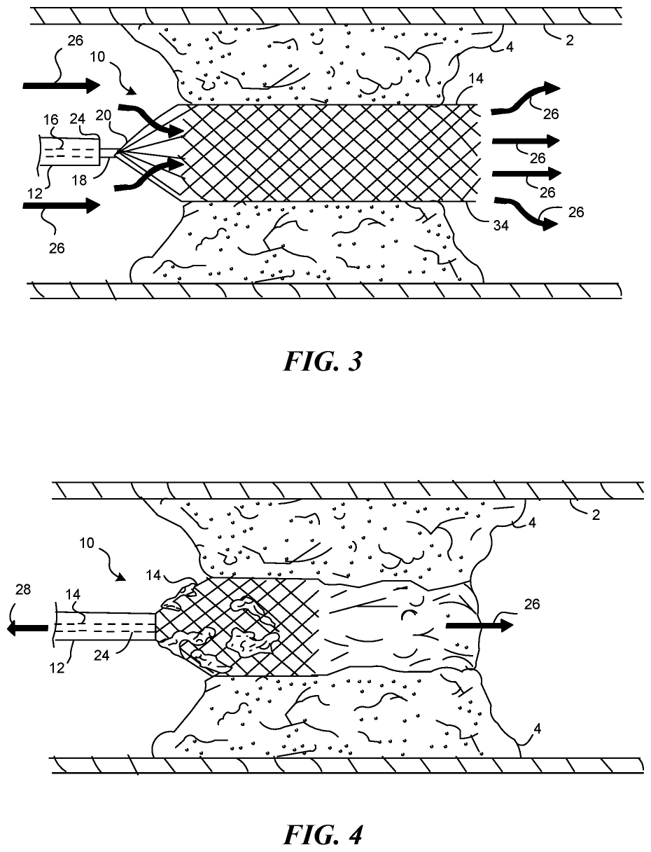

As shown in FIG. 3, when catheter 12 is retracted sufficiently to allow expansion of the entire length of the flow restoration member 14, fluid or blood may enter the open, proximal portion 20 of the flow restoration member 14 in the direction of arrows 30, flow through the hollow interior of the flow restoration member 14, and exit through a open, distal portion 34 of the flow restoration member 14. Thereby, allowing for a rapid restoration of blood flow through the lumen 2.

As shown in FIG. 4, the pusher 16 is then retracted relative to the catheter in the direction of the arrow 28, thereby pulling the length of the flow restoration member 14 through the occlusive material 4. The pusher 16 is retracted such that the flow restoration member 14 is pulled towards the distal end 24 of the catheter 12 and back into the lumen 22 of the catheter 12. As the flow restoration member 14 is pulled through the occlusive material 4, the occlusive material 4 engaged with the flow restoration member 14 is also pulled along and removed. Hence, while restoring flow through the lumen 2, the flow restoration member 14 may also function to remove or extract at least a portion of the occlusive material 4 from the lumen 2. Finally, the flow restoration member 14 and the engaged occlusive material 4 is pulled back into the lumen 22 of the catheter 12 and the system 10 is withdrawn from the patient.

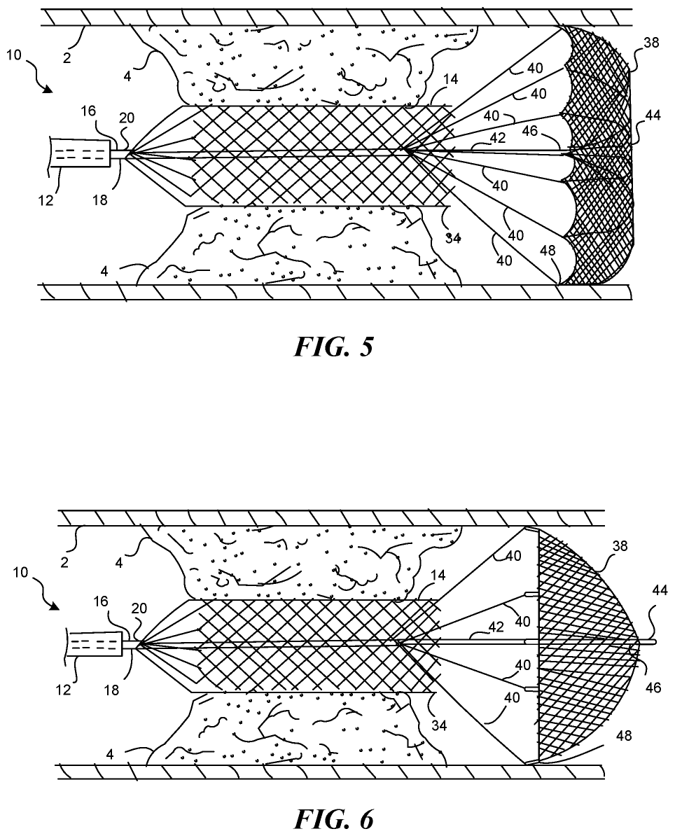

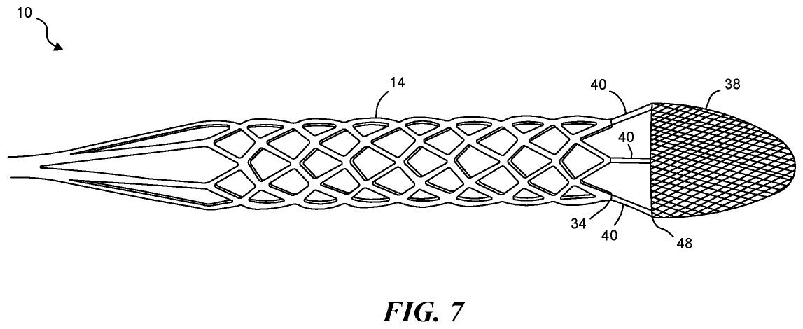

In one embodiment of the present invention, as shown in FIGS. 5-7, the occlusion management system 10 may further employ an extraction member 38 for extraction or removal of the occlusive material 4, such as an embolus. The extraction member 38 may have an umbrella-like configuration, as shown in FIG. 5; a conical configuration, as shown in FIG. 6; or a cup-like configuration, as shown in FIG. 7. The extraction member 38 expands from a compressed diameter to an expanded diameter that is greater than a diameter of the expanded flow restoration member 14 and approximately equal to a diameter of the lumen 2.

The extraction member 38 may be attached directly to the flow restoration member 14 or to a separate structure that is deployed through the flow restoration member 14 either before or after deployment of the flow restoration member 14. For example, as shown in FIGS. 5 and 6, a distal portion 44 of the extraction member 38 may be attached to a distal end 46 of a delivery element 42. The delivery element 42 may be formed of a separate, transposable element that is located within a lumen of the pusher 16. One or more tethers 40 may statically attach a proximal periphery 48 of the extraction member 38 to the delivery element 42 proximally of the distal end 46 of the delivery element 42. The tethers 40 facilitate compression and retraction of the extraction member 38 back into the catheter 12. Alternatively, the tethers 40 may be transposable independent of the delivery element 42. For example, the tethers 40 may be attached to a coaxial tube located within the lumen of the pusher 16 around the delivery element 42.

In operation, the extraction member can be deployed either prior to complete deployment of the flow restoration member 14 or after complete deployment of the flow restoration member 14.

In certain embodiments, as shown in FIG. 25, the extraction member 38 is a balloon 56 that is attached to a distal end 46 of a delivery element 42. The delivery element 42 has a lumen formed therethrough for inflation and deflation of the balloon 56. The balloon 56 having a diameter that is substantially equal to or greater than a diameter of the vessel 2.

In certain other embodiments, as shown in FIG. 26, the extraction member 38 is formed by a malecot-type formation of the distal end 46 of the delivery element 42. The malecot-type formation may be covered with a fabric, polymer, or braided covering. The malecot-type formation has a diameter that is substantially equal to or greater than a diameter of the vessel 2.

In certain other embodiments, as shown in FIG. 29 the extraction member 38 is formed of a braided structure having a disc-like form that is attached to a distal end 46 of a delivery element 42. The disc-like structure has a diameter that is substantially equal to or greater than a diameter of the vessel 2.

In one embodiment of the present invention, as shown in FIG. 7, the delivery element 42 is not employed in the system 10 and extraction member 38 is attached directly to the flow restoration member 14 by the tethers 40. More particularly, proximal ends of the tethers 40 are attached to the distal portion 34 of the flow restoration member 14 and distal ends of the tethers 40 are attached to the proximal periphery 48 of the extraction member 38.

In operation, after the catheter 12 is advanced through the occlusive material 4 until a distal end 24 of the catheter 12 passes entirely through the occlusive material 4, the catheter 12 is then retracted relative to the pusher 16. As the extraction member 38 is exposed from the retracting distal end 24 of the catheter 12, the extraction member 38 radially expands distally of the occlusive material 4. As the catheter 12 is further retracted, the flow restoration member 14 radially expands within the occlusive material 4.

After complete expansion of the flow restoration member 14, the pusher 16 is retracted relative to the catheter, thereby pulling the flow restoration member 14 through the occlusive material 4 and pulling the extraction member 38 into and around the occlusive material 4. The occlusive material 4 is thereby captured within the extraction member 38. Retraction of the pusher 16 is continued until the flow restoration member 14 and extraction member 38 with captured occlusive material 4 are pulled back into the lumen 22 of the catheter 12. The system 10 is then withdrawn from the patient.

The extraction member 38 may be formed at least in part by a braid of filaments or fabricated by methods known in the art of stent manufacturing including but not limited to conventional machining, laser cutting, electrical discharge machining (EDM) and photo-chemical etching.

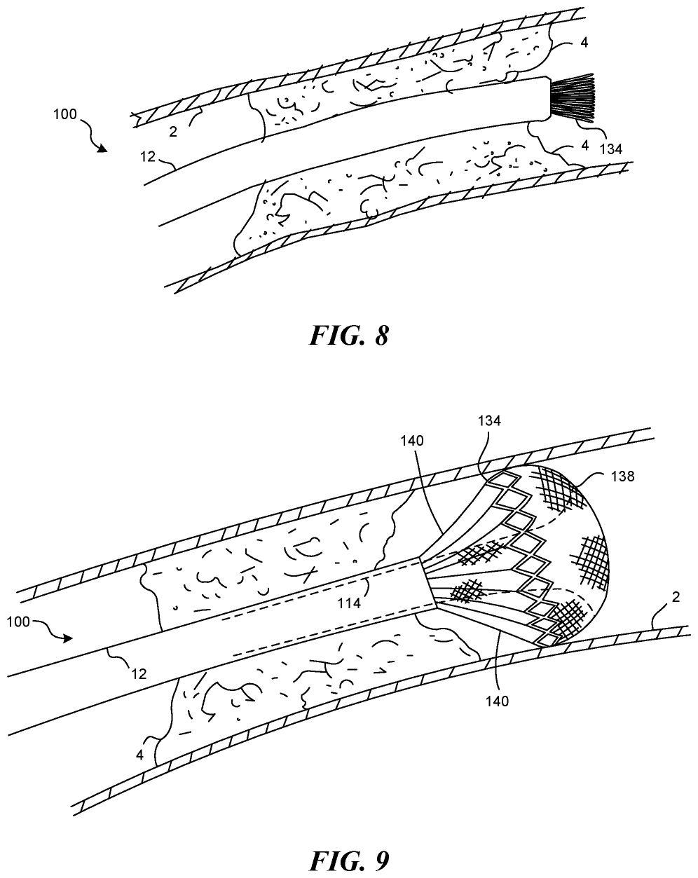

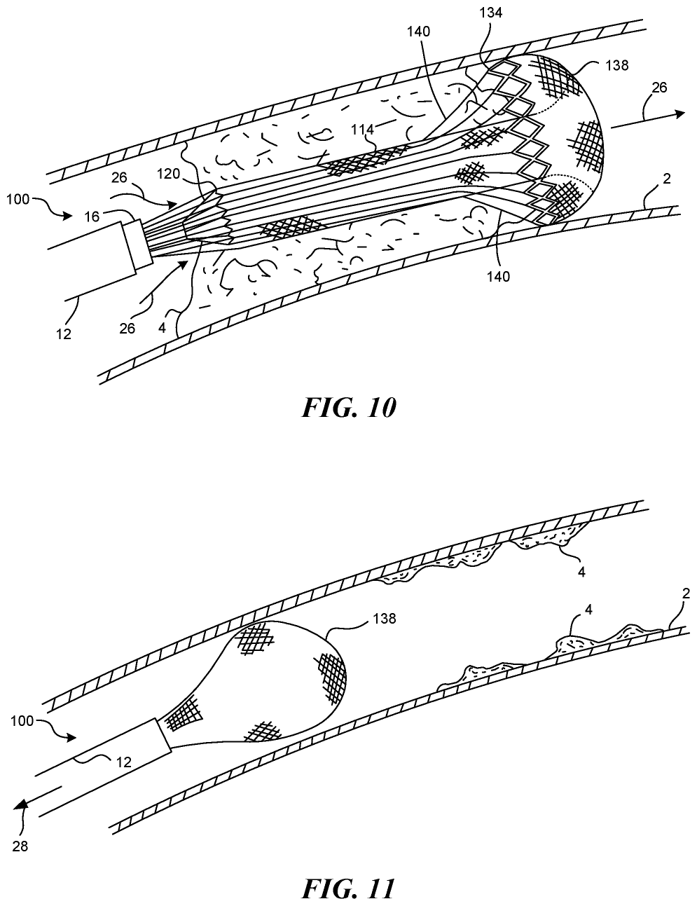

In one embodiment of the present invention, as shown in FIGS. 8-11 , the flow restoration member and the extraction member of the occlusion management system 100 are formed of a substantially continuous structure. For example, as shown in FIG. 10, a distal portion 134 of a flow restoration member 114 is biased to evert to a relaxed state that turns in a proximal direction back towards a proximal portion 120 of the flow restoration member 114, thereby forming an extraction member 138. One or more tethers 140 are eccentrically coupled or attached to the distal portion 134 of a flow restoration member 114. In certain embodiments, a radially expandable connector member 150 may hold ends of the filaments that may be present at the distal portion 134 of a flow restoration member 114.

Proximal ends of the tethers 140 may extend proximally within the lumen 22 of the catheter 12 and may be manipulated by a physician in order to facilitate the formation of the everted distal portion 134 and extraction member 138 of the flow restoration member 114. In certain embodiments, the tethers 140 do not extend to a proximal end of the system 100 but rather are connected to an elongate retraction member that in turn extends proximally for manipulation by a physician. As shown in FIG. 10, the tethers 140 may further function to cut through the occlusive material 4 as the extraction member 138 is formed or when the pusher 16, the flow restoration member 114, and the extraction member 138 are retracted relative to the catheter 12.

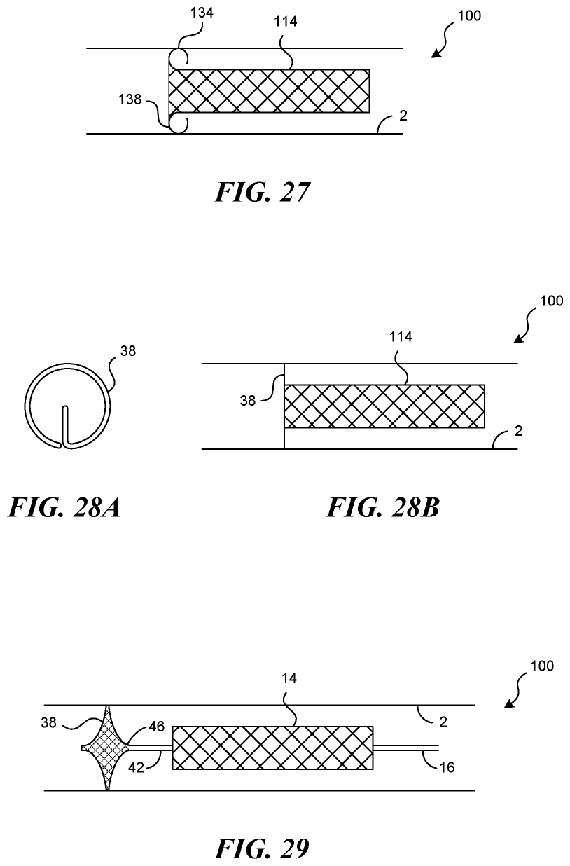

In certain embodiments, as shown in FIG. 27, the flow restoration member 114 having everted distal portion 134 need not necessarily employ the tethers 140.

In certain other embodiments, as shown in FIGS. 21-24, 28A, and 28B, the mesh or scaffold structure forming the flow restoration member 114 employs an enlarged diameter distal portion 134 that does not necessarily evert. For example, FIG. 21 shows a partially deployed and FIG. 23 shows completely deployed flow restoration member 114 having a flared or expanded distal portion 134. FIG. 24 shows the flow restoration member 114 having a bulbous, expanded distal portion 134 which may or may not employ a guide wire passage through a distal end.

In certain other embodiments, as shown in FIG. 28A and 28B, the extraction member 38 is a wireform attached to the delivery element, such as delivery element 42 described above, or alternatively attached directly to the flow restoration member 114 to form an expanded distal portion 134 of the flow restoration member 114. The wire form may also be covered with a braid. As shown in FIGS. 8-11, operation of the occlusion management system 100 is substantially the same as described above regarding the occlusion management system 10 employing the extraction member 38.

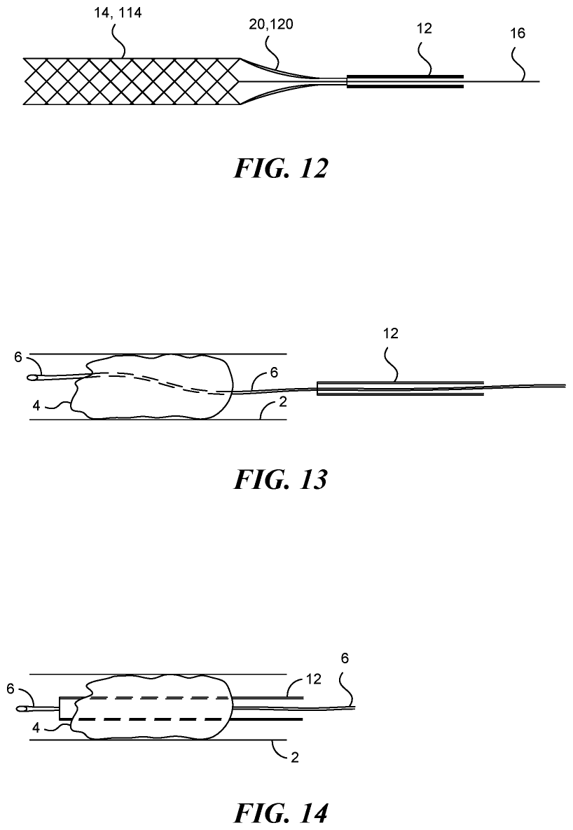

In one embodiment of the present invention, as shown in FIG. 12, the pusher 16 may be formed of a wire, tube, or catheter.

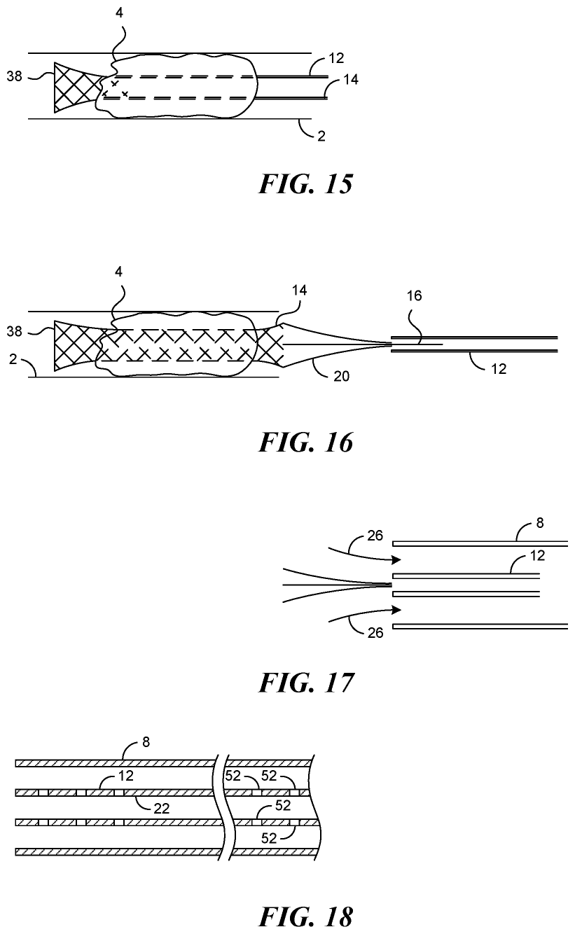

In one embodiment of the present invention, as shown in FIGS. 13-19, a method for operation of system 10, 100 is shown. First, retrieval of occlusive matter 4 includes first advancing a guidewire 6 through a lumen 2 to the site of the occlusive material 4 and through the occlusive material 4. The catheter 12 is then advanced over the guidewire 6 to the site of the occlusive material 4 and through the occlusive material 4, as shown in FIGS. 13 and 14. The guidewire 6 is withdrawn from the patient. As shown in FIG. 15, the catheter 12 is then retracted relative to the pusher 16, thereby allowing the flow restoration member 14 to expand to a more relaxed state and engage the occlusive material 4.

In certain embodiments, as shown in FIG. 17, the catheter 12 may be passed through a lumen of a sheath 8. The sheath 8 may function to provide suction, vacuum, or irrigation, in the direction of arrows 26, within the lumen 2 near the site of the occlusive material 4. Alternatively, as shown in FIG. 18, one or more holes 52 may be formed in the catheter 12 so that the suction, vacuum, or irrigation may originate from a proximal end of the catheter 12 and be simultaneously generated through the proximal portions of both the lumen 22 of the catheter 12 and the lumen of the sheath 8.

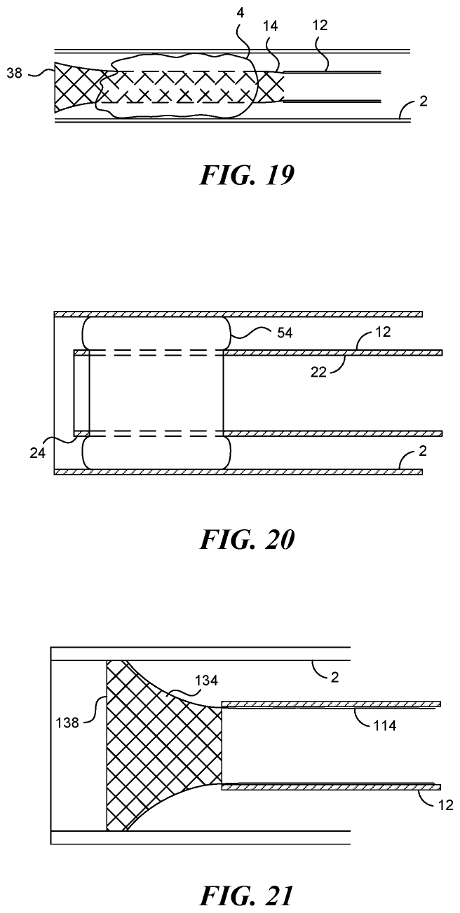

With the assistance of such suction, vacuum, or irrigation, as shown in FIG. 19, it may be possible for the flow restoration member 14 to sufficiently engage the occlusive material 4 such that the occlusive material 4 is released from the lumen 2 and can be extracted in substantially its entirety from the lumen 2 of the patient.

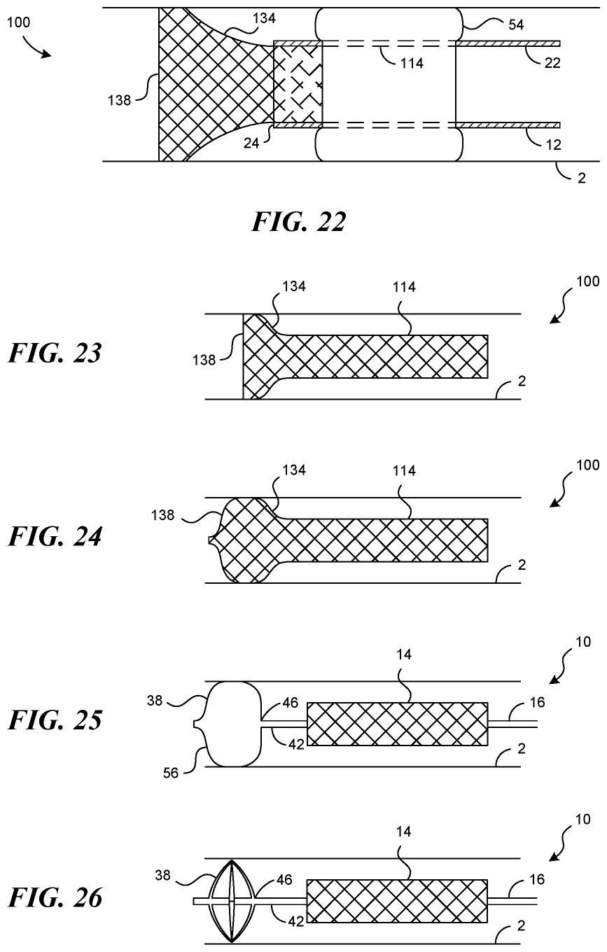

In one embodiment of the present invention, as shown in FIG. 20, in order to further assist in the generation and efficacy of such suction, vacuum, or irrigation, an annular balloon 54 may be attached to an exterior of the catheter 12 near the distal end 24 of the catheter 12. The balloon 54 is sized so as to contact a circumference of an interior surface of the lumen 2. Accordingly, the balloon 54 provides a seal against the flow of fluid, such as blood, through the lumen 2 and enhances the efficacy of the suction, vacuum, or irrigation. FIG. 22 shows the flow restoration member 114 of FIG. 23 being deployed through a catheter 12 having an inflated balloon 54 near the distal end 24 of the catheter 12. In order to inflate and deflate the balloon 54, inflation lumens may be formed within the wall of the catheter 12 according to techniques known in the art.

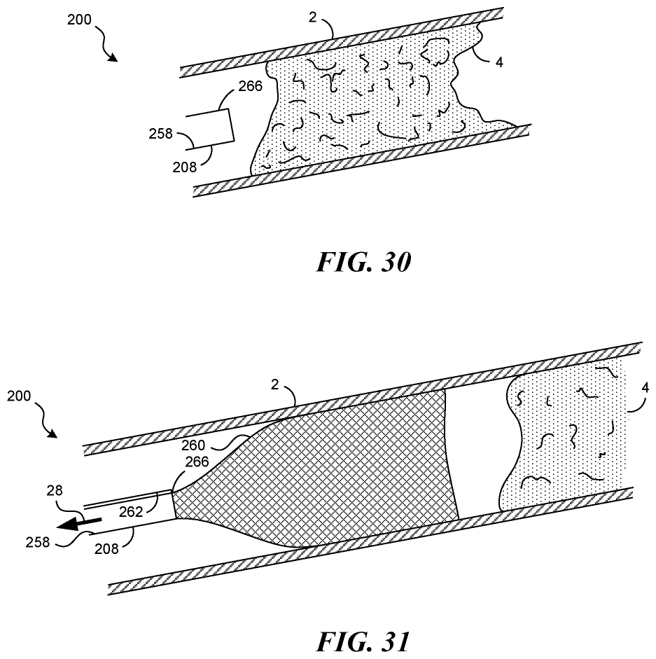

In one embodiment of the present invention, as shown in FIGS. 30-35, an occlusion management system 200 employs a flow restoration member 214, such as that described above with respect to the flow restoration members 14 or 114 that is advanceable through a proximal capture member 260.

The proximal capture member 260 is radially expandable from compressed delivery state within a lumen 258 of a sheath 208, to a radially expanded, minimum energy state having a generally cylindrical or tubular shape. When in the expanded minimum energy state, the proximal capture member 260 may have a diameter that is larger or substantially equal to the diameter of the patient's lumen 2 in which the system 200 will be employed.

The proximal capture member 260 is attached to a capture member pusher 262 that is also inserted through the lumen 258 of the sheath 208. The proximal capture member 260 may be formed of a mesh or scaffold. The mesh or scaffold may be formed at least in part by a braid of filaments or fabricated by methods known in the art of stent manufacturing including but not limited to conventional machining, laser cutting, electrical discharge machining (EDM) and photo-chemical etching.

The flow restoration member 214 is attached to the pusher 16 and the flow restoration member 214 and the pusher 16 are positioned within the lumen 22 of the catheter 12. The catheter 12 is, in turn, positioned within a lumen of the proximal capture member 260. A diameter of the proximal capture member 260 may be approximately equal to or greater than a diameter of the lumen 2.

In operation, the capture member pusher 262 and attached proximal capture member 260 are inserted into the lumen 258 of the sheath 208. A guidewire may be advance through the occlusion material 4, such as a thrombus or embolus. The sheath 208 is then advanced over the guidewire to a position proximal of the occlusion material 4. The guidewire may but need not necessarily be retracted at this time.

As shown in FIGS. 30 and 31, the sheath 208 is retracted, in the direction of arrow 28, proximally relative to the capture member pusher 262, thereby exposing the proximal capture member 260 at a distal end 266 of the sheath 208 and allowing the proximal capture member 260 to radially expand from its collapsed state within the lumen 258 of the sheath 208.

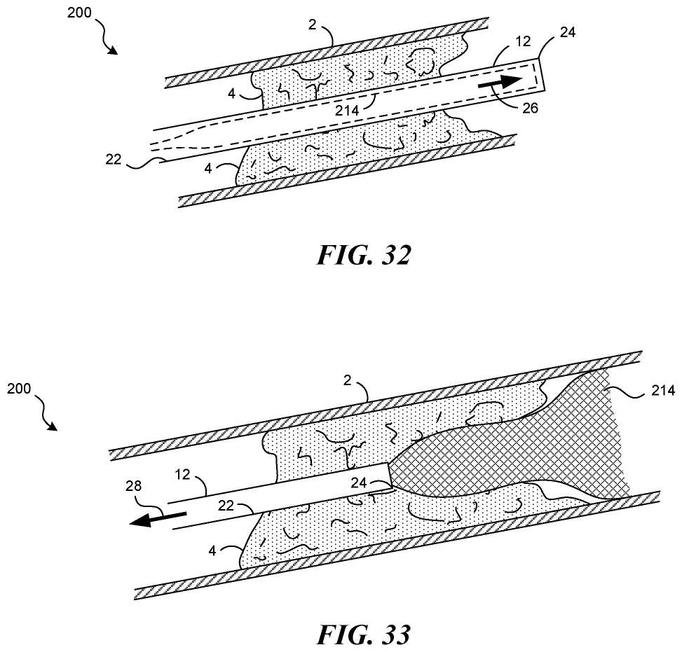

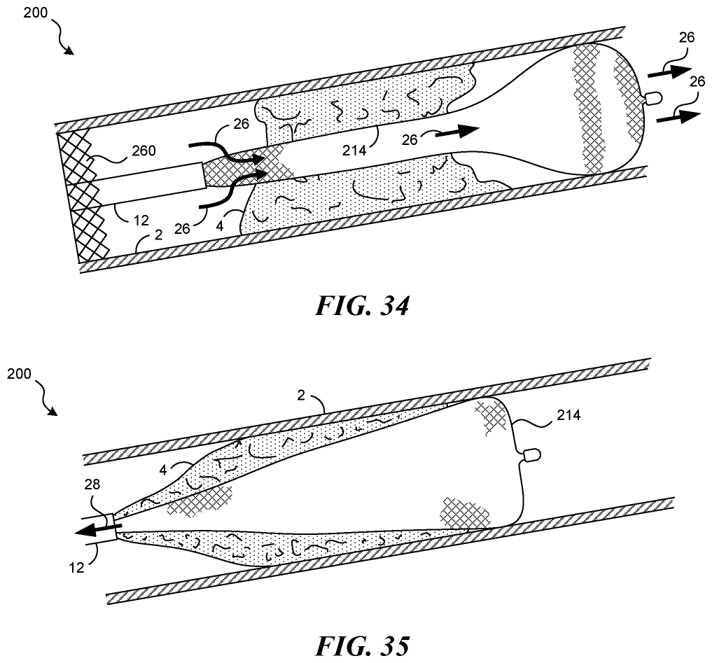

The pusher 16 and attached flow restoration member 214 are then inserted into the lumen 22 of the catheter 12. As shown in FIG. 32, the catheter 12 is then advanced through the lumen 258 of the sheath 208 and the lumen of the proximal capture member 260 until a distal end 24 of the catheter 12 is positioned distally of the occlusive material 4. As shown in FIGS. 33 and 34, the catheter 12 is then retracted, in the direction of arrow 28, proximally relative to the flow restoration member 214, thereby exposing the flow restoration member 214 and allowing the flow restoration member 214 to radially expand from its collapsed state within the lumen 22 of the catheter 12.

As shown in FIG. 35, after complete expansion of the flow restoration member 214, the pusher 16 is retracted relative to the catheter 12, thereby pulling the flow restoration member 214 through the occlusive material 4 and pulling an extraction member, if present, into and around the occlusive material 4. The occlusive material 4 is thereby captured within the flow restoration member 214 and extraction member, if present. Retraction of the pusher 16 is continued until the flow restoration member 214 and extraction member, if present, with captured occlusive material 4 are pulled at least partially back into the lumen 22 of the catheter 12. The catheter 12 and the flow restoration member 214 and extraction member, if present, with captured occlusive material 4 are then pulled back into the lumen 264 of the proximal capture member 260. The proximal capture member 260 is then pulled back into the lumen 258 of the sheath 208. The system 200 is then withdrawn from the patient.

The order of deployment of the proximal capture member 260 and flow restoration member 214 as described above may be reversed as seen fit by the physician. Furthermore, therapeutic agent(s) such as thrombolytics or anticoagulants may be infused through the lumen 258 of the sheath 208 or lumen 22 of catheter 12 during the course of the procedure.

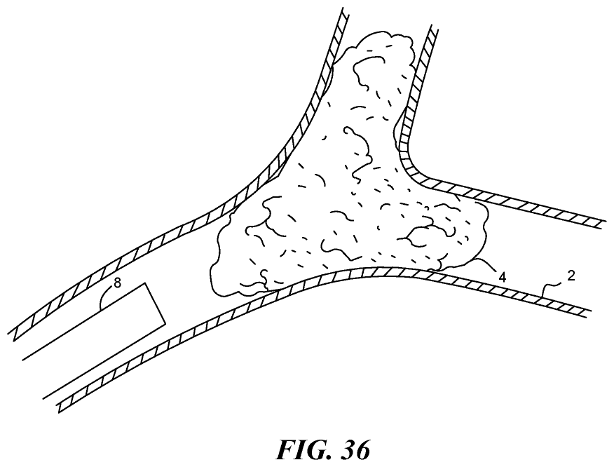

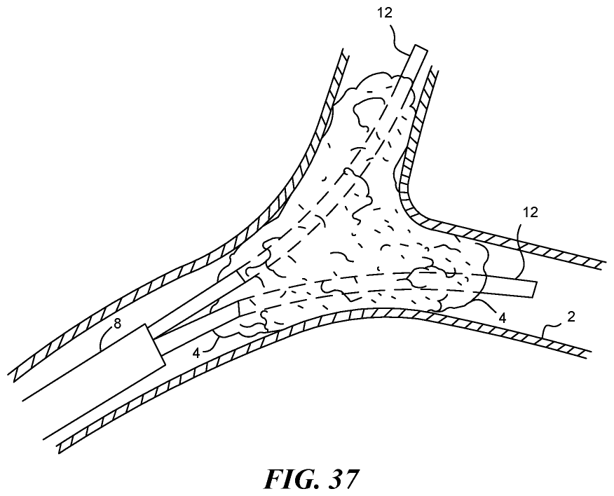

In one embodiment of the present invention, the occlusion management systems 10, 100, 200 is configured for removal of at least a portion of the occlusive material 4, such as an embolus or thrombus, that is located at a bifurcation, trifurcation or multi-lumen plexus of the lumen 2, such as a blood vessel. By way of example, as shown in FIGS. 36 and 37, a sheath 8, through which multiple catheters 12 are inserted, is advanced through the lumen 2 to the bifurcation at which occlusive material 4 is present. The catheters 12 are independently advanced distally from the sheath 8 through the occlusive material 4 within the different lumens 2 of the bifurcation. Flow restoration and extraction of the occlusive material 4 is conducted as described above.

In certain embodiments of the present invention, the flow restoration member 14, 114, 214, extraction member 38, 138, and the proximal capture member 260 may comprise a braided mesh of filaments or wires 70. The braids for the mesh components may have a generally constant braid angle over an entire length of the member or may be varied to provide different zones of pore size and radial stiffness.

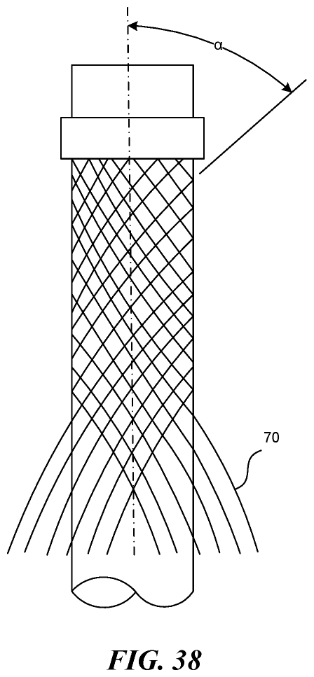

The braided mesh may be formed over a mandrel as is known in the art of tubular braid manufacturing. A braid angle a (alpha), shown in FIG. 38, may be controlled by various means known in the art of filament braiding. In certain embodiments, the braid angle a is, for example, between about 45 degrees and about 60 degrees. The tubular braided mesh may be further shaped using a heat setting process. As known in the art of heat setting nitinol wires, a fixture, mandrel or mold may be used to hold the braided tubular structure in its desired configuration then subjected to an appropriate heat treatment such that the resilient filaments of the braided tubular member assume or are otherwise shape-set to the outer contour of the mandrel or mold.

In certain embodiments, the filamentary elements of the mesh member may be held by a fixture configured to hold the member in a desired shape and heated to about 475-525 degrees Celsius for about 5 to 30 minutes to shape-set the structure. In certain embodiments, the braid may be a tubular braid of fine metal wires 70 such as Nitinol, platinum, cobalt-chrome alloys, 35N LT, Elgiloy, stainless steel, tungsten or titanium.

In certain embodiments, the member can be formed at least in part from a cylindrical braid of elastic filaments. Thus, the braid may be radially constrained without plastic deformation and will self-expand on release of the radial constraint to an unrestrained diameter or diameter at its lowest energy state. Such a braid of elastic filaments is herein referred to as a “self-expanding braid.”

In certain embodiments, the thickness of the braid filaments is less that about 0.5 millimeters. In certain embodiments, the braid may be fabricated from wires 70 with diameters ranging from about 0.015 millimeters to about 0.40 millimeters. In certain embodiments, the braid may be fabricated from wires with diameters ranging from about 0.02 millimeters to about 0.15 millimeters.

In certain embodiments, the member has a high braid angle zone where the braid angle a is greater than about 60 degrees. More particularly, the higher braid angle portion or zone may have a braid angle a that is between 60 and 80 degrees. The high braid angle portion may have higher radial stiffness that may provide, for example, improved extraction of occlusive material 4. Furthermore, as the member is retracted the portion of the member with a high braid angle elongates to a greater amount relative to the remainder of the member, thereby providing a longer surface for retraction through the occlusive material.

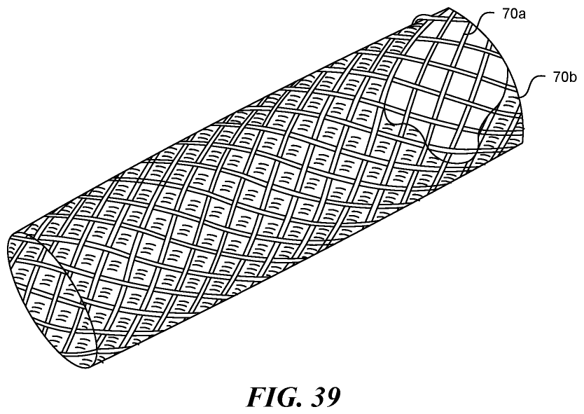

In certain embodiments, the system may comprise a braided member where the braid is formed from a mixture of more than one diameter wire 70, as shown in FIG. 38. A braid showing two wire diameters, wire 70a and wires 70b having a smaller diameter than the diameter of the wires 70a, is shown in FIG. 39.

A braided member may also comprise a plurality of layers. In certain embodiments, the system may comprise a braided member where the braid configuration changes over the length of the member forming a tubular structure with two or more zones of different braid. The parameters that may be changed to manipulate the braid include but are not limited to braid angle a, combinations of different diameters of wire 70 (e.g. a combination of small and large diameters) and wire loading (e.g. alternating wire size in a 1 by 1 or 2 by 2 pattern). Changing the braid parameters allows for zones of different mechanical properties (e.g. radial stiffness and compliance) along one continuous braid. In certain embodiments, the member may have one zone with a braid angle a between about 35 degrees and 55 degrees and another zone with a braid angle a between about 50 degrees and 70 degrees. In certain embodiments, the member may have one zone with a radial stiffness that is at least about 25% greater than the radial stiffness of a second zone.

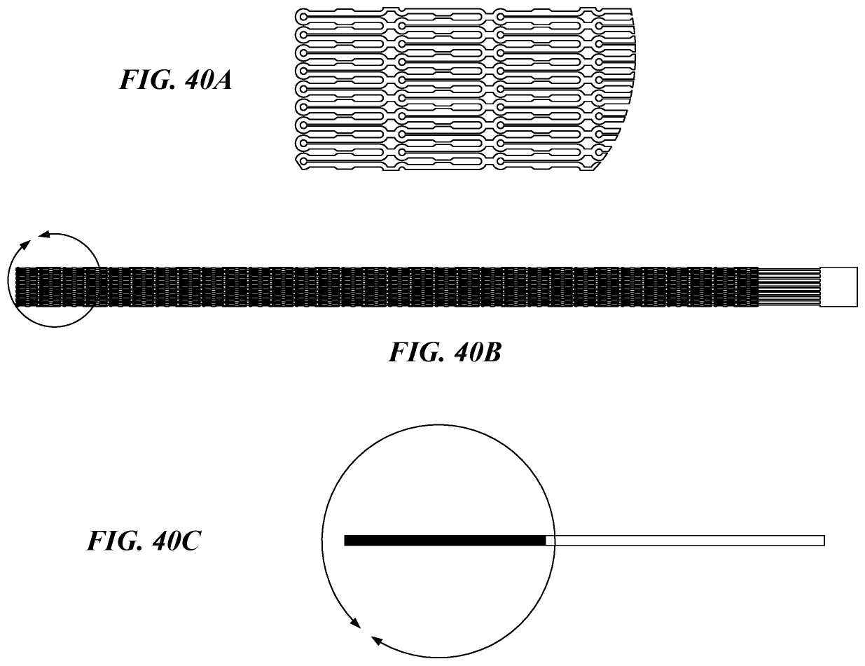

In one embodiment of the present invention, as shown in FIGS. 40A-40C, the flow restoration member may be formed by machining or laser cutting a stent-like pattern either directly in a tube or in a flat sheet that is subsequently formed into a tube. The sheet may be rolled or otherwise formed into a generally tubular configuration and then welded, soldered or joined in order to fix the tubular shape. FIG. 40A shows an exemplary flat pattern. FIG. 40B shows the tube form of the stent-like pattern and FIG. 40C shows the stent-like tube attached to the distal end of a pusher or delivery element. In certain other embodiments, as shown in FIG. 27, the extraction member 138 is a braided structure extension of flow restoration member 114 that has been everted and curled back on itself forming an expanded distal portion. In any of the above described embodiments, the system 10, 10, 200 may include additional devices or components to facilitate thrombus maceration or disruption including but not limited to mechanical maceration members (auger, drill bit, screw, impellor, burr, pick, etc.), vibration members, ultrasonic energy, radiofrequency energy, microwave energy, thermal energy, cavitiation, flow jets or perfusion apparatus. For example, in certain embodiments, the system 10, 100, 200 may comprise a boring member to facilitate penetration of the occlusive material 4. In certain embodiments, the system 10, 100, 200 may comprise an auger device to facilitate retraction of the occlusive material 4, such as thrombus along a central path coaxial with the flow restoration member 14, 114, 214.

In any of the above described embodiments, the system 10, 100, 200 may include a drug or bioactive agent to enhance the thrombus extraction performance and/or reduce the propensity to produce clotting. In certain embodiments, the system 10, 100, 200 and more particularly the flow restoration member 14, 114, 214, extraction member 38, 138, and the proximal capture member 260 may employ textures, surface features, coatings, or the like to enhance the engagement and/or attachment of the occlusive material 4, such as thrombus. In certain embodiments, the device may include an antiplatelet agent, a lytic agent or an anticoagulant.

In any of the above described embodiments, a delivery system may be provided or integrated into the catheter 10 and/or sheath 8, 208. The delivery system may include an introducer sheath for access into the appropriate vein such as the subclavian vein, jugular vein, femoral vein or radial vein. In certain embodiments, the catheter 10 and/or sheath 8, 208 may be placed through the introducer sheath to pass through the access vein such as the right subclavian vein or jugular vein into the superior vena cava through the right atrium through the tricuspid valve, through the right ventricle, through the pulmonic valve, to thrombus or occlusive embolus situated in the pulmonary artery or branches of the pulmonary artery. In some embodiments, the catheter 10 and/or sheath 208 may be placed through the introducer sheath to pass through the access vein such as the femoral vein into the inferior vena cava through the right atrium through the tricuspid valve, through the right ventricle, through the pulmonic valve, to thrombus or occlusive embolus situated in the pulmonary artery or branches of the pulmonary artery.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A method for removing occlusive material from a peripheral blood vessel in a patient, the method comprising:
advancing an introducer sheath through a peripheral vessel and toward occlusive material;
positioning a distal end of the introducer sheath proximal of the occlusive material, wherein a proximal capture member is contained within a lumen of the introducer sheath, the proximal capture member having a tapered proximal end portion and an open distal end sized to engage an inner wall of the vein;
distally advancing an occlusion removal device from the lumen of the introducer sheath and through the occlusive material, wherein the occlusion removal device is contained within a distal end portion of the catheter, wherein the occlusion removal device comprises a first portion and a second portion extending from the first portion, and wherein the second portion comprises a braided material;
retracting the catheter for releasing the occlusion removal device and allowing the occlusion removal device to self-expand such that the first portion is positioned distally from the occlusive material and at least a portion of the second portion extends distally from the first portion;
withdrawing the occlusion removal device through the vessel such that the first portion of the occlusion removal device engages the occlusive material and then the braided material of the second portion of the occlusion removal device contains at least a portion of the occlusive material;
further withdrawing the occlusion removal device through the vessel with at least a portion of the occlusive material contained in the braided material of the second portion of the occlusion removal device and through a central opening in the proximal capture member and into the lumen of the sheath; and
collapsing the proximal capture member into the lumen of the sheath.

2. The method of claim 1, wherein the occlusion removal device comprises a tubular member.

3. The method of claim 1, wherein the first portion of the occlusion removal device comprises a ringed-shape member configured to expand to an inner surface of the peripheral vessel and the second portion of the occlusion removal device comprises a braided mesh extending from the ring-shaped member, and wherein the braided mesh is configured to contain at least a portion of the occlusive material after the ring-shaped member passes through the occlusive material.

4. The method of claim 3, wherein the first portion and the second portion of the occlusion removal device form a continuous structure.

5. The method of claim 1, wherein the occlusion removal device comprises a tubular member that is everted to form a distal end of the second portion of the occlusion removal device.

6. The method of claim 5, further comprising a pusher coupled to a periphery of the tubular member.

7. The method of claim 6, wherein at least one tether extends between the pusher and the periphery of the tubular member.

8. The method of claim 1, wherein the first portion of the occlusion removal device comprises a scaffold configured to expand to an inner surface of the peripheral vessel and the second portion of the occlusion device comprises a braided mesh configured to contain at least a portion of the occlusive material after the scaffold passes through the occlusive material.

9. A method for removal of an occlusive thrombus from a peripheral blood vessel for improving blood flow through the blood vessel, the method comprising:
advancing an introducer sheath through a peripheral blood vessel;
positioning a distal end of the introducer sheath proximal of an occlusive thrombus, wherein a proximal capture member is contained within a lumen of the introducer sheath, the proximal capture member formed with a mesh structure having a tapered proximal end portion and an open distal end, wherein the open distal end is shaped to conform to an inner wall of the peripheral blood vessel when in an expanded configuration;
advancing an occlusion removal device through the lumen of the introducer sheath and through a central opening in the proximal capture member, wherein the occlusion removal device comprises a tubular member adapted to engage the occlusive thrombus;
deploying at least a portion of the tubular member distal to the occlusive thrombus;
deploying the proximal capture member;
withdrawing the tubular member and thereby engaging the occlusive thrombus such that the tubular member contains at least a portion of the occlusive thrombus;
further withdrawing the tubular member with captured occlusive thrombus through the proximal capture member and collapsing the tubular member into the lumen of the introducer sheath; and
collapsing the proximal capture member into the lumen of the introducer sheath.

10. The method of claim 9, wherein a distal portion of the tubular member is everted relative to a central axis to form an extraction member configured to capture and remove the occlusive thrombus.

11. The method of claim 9, wherein the tubular member includes a first end portion adapted for engaging an inner surface of the peripheral blood vessel during withdrawal of the tubular member through the peripheral blood vessel.

12. The method of claim 11, wherein the first end portion comprises a relatively rigid ring-shaped structure.

13. The method of claim 11, wherein the tubular member includes a second end portion configured to define a flow restoration member.

14. The method of claim 13, wherein the tubular member everts from the flow restoration member to the extraction member in an expanded state.

15. The method of claim 13, wherein a pusher is coupled to a periphery of the flow restoration member.

16. A method for removing a thrombus from a peripheral blood vessel in a patient's body, the method comprising:
identifying a thrombus in a peripheral blood vessel;
advancing a guidewire through the thrombus
advancing a catheter over the guidewire, wherein a thrombus removal device is contained within a lumen of the catheter, the thrombus removal device comprising a ring-shaped member configured to expand to an inner surface of the peripheral blood vessel and an extraction member extending from the ring-shaped member;
retracting the catheter for allowing the thrombus removal device to self-expand in the peripheral blood vessel such that the ring-shaped member engages the inner surface of the peripheral blood vessel;
pulling the thrombus removal device in a proximal direction such that the ring-shaped member passes along the thrombus and the extraction member captures material from the thrombus and thereby forms a passageway for the flow of blood;
further withdrawing the thrombus removal device through a central opening in a proximal capture member and into a sheath, the proximal capture member comprising a mesh structure having an open distal end sized to conform to an inner wall of the peripheral blood vessel; and
removing the guidewire, the sheath, the proximal capture member, the catheter, and the tubular member from the patient's body, thereby restoring blood flow through the peripheral blood vessel.

17. The method of claim 16, wherein the extraction member has a closed distal end for capturing and pulling thrombus material into the sheath.

18. The method of claim 17, wherein the ring-shaped member and the extraction member form a continuous structure.

19. The method of claim 18, wherein the closed distal end of the extraction member comprises an everted portion.

20. The method of claim 16, wherein the ring-shaped member is a self-expanding structure and the extraction member is a mesh structure extending distally therefrom.
